(19) United States
(12) Patent Application Publication
(10) Pub. No.: US 2008/0241192 A1
(43) Pub. Date: Oct. 2, 2008

(54) Vaccination against multiple serotypes of pasteurella multocida

(75) Inventors: Mahesh Kumar, Fort Dodge, IA (US); Christine Anschutz, Jefferson, IA (US); Jean Q. Tian, Fort Dodge, IA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/079,117

(22) Filed: Mar. 25, 2008

Related U.S. Application Data
(60) Provisional application No. 60/919,997, filed on Mar. 26, 2007.

(51) Int. Cl. A61K 39/102

(52) U.S. Cl. 424/255.1

(57) ABSTRACT

The present invention provides methods for inducing cross-protective immunity against virulent strains of P. multocida in animals such as cattle and poultry. The methods of the invention include administering to an animal a mutant P. multocida strain, whereby the mutant P. multocida strain induces cross-protective immunity against one or more virulent P. multocida strains having serotypes that are different from the serotype of the mutant P. multocida strain. The mutant P. multocida strain will preferably contain one or more mutations that cause the cells to be acapsular and/or attenuated. Exemplary mutations include, e.g., mutations that impair the expression of one or more genes in the P. multocida capsule biosynthetic operon (e.g., phyB, phyA, hyaE, hyaD, hyaC, hyaB, hexD, hexC, hexB, and/or hexA).

### CROSS-REFERENCE TO RELATED APPLICATIONS

This application claims priority from copending U.S. provisional application No. 60/919,997, filed Mar. 26, 2007, the entire disclosure of which is hereby incorporated by reference.

### BACKGROUND OF THE INVENTION

1. Field of the Invention

The present invention relates to vaccines that are effective against virulent strains of P. multocida.

2. Background Art

Pasteurella multocida is a bacterial pathogen. P. multocida is the causative agent of multiple diseases in several species of animals. For example, P. multocida is known to cause hemorrhagic septicemia in ungulates, atrophic rhinitis in swine, and fowl cholera in wild and domestic birds.

P. multocida strains generally express a polysaccharide-containing capsule on their surface. Mutant P. multocida that lack a capsule (“acapsular” mutants) have been shown to be avirulent and/or attenuated. (Watt et al., FEMS Microbiol. Lett. 225:9-14 (2003)). Acapsular mutants have been produced by subculturing P. multocida on laboratory growth media (Watt et al., FEMS Microbiol. Lett. 225:9-14 (2003)), by enzymatic depolymerization of the capsule polysaccharides (Jacques et al., Infect. Immun. 61:4785-4792 (1993)), and by mutating genes involved in capsule biosynthesis. (Chung et al., Infect. Immun. 69:2487-2492 (2001), U.S. Patent Appl. Publ. No. 2005/0106185).

P. multocida strains can be classified according to serogroup and serotype. There are five different serogroups, designated A, B, D, E and F, based on capsular antigens. There are sixteen different serotypes, designated 1 through 16, based on somatic lipopolysaccharide (LPS) antigens. Typically, P. multocida strains are designated according to both serogroup and serotype in terms such as “A:1,” “A:3,” “A:4,” etc. For simplicity, the serogroup:serotype designation of a particular P. multocida strain will sometimes be referred to herein as simply the “serotype” of that strain.

U.S. Patent Appl. Publ. No. 2005/0106185 refers to P. multocida acapsular mutants of serotype A:3 that contain a deletion of part of the hyaE gene. The hyaE deletion mutants are said to be acapsular and attenuated. U.S. Patent Appl. Publ. No. 2005/0106185 refers to the use of A:3 P. multocida hyaE deletion mutants in vaccine preparations. There is no suggestion, however, that acapsular P. multocida mutants of serotype A:3 could provide protection against virulent strains of P. multocida having a different serotype.

There exists a need in the art for live, attenuated P. multocida vaccines and vaccination methods that provide protection, not only against infection by virulent strains of P. multocida having the same serotype as the attenuated P. multocida, but also against infection by virulent strains of P. multocida having different serotypes, i.e., cross-protective immunity. For example, there exists a need in the art for vaccination methods in which an attenuated strain of P. multocida provides cross-protective immunity against infection by virulent strains of P. multocida having serotypes that are different from the serotype of the attenuated P. multocida strain.

### BRIEF SUMMARY OF THE INVENTION

The present invention satisfies the aforementioned need in the art by providing methods for inducing cross-protective immunity against a virulent strain of P. multocida having a particular serotype by administering to an animal a mutant P. multocida having a different serotype. The mutant P. multocida strain will preferably contain one or more mutations that cause the cells to be acapsular and/or attenuated. Exemplary mutations include, e.g., mutations that impair the expression of one or more genes in the P. multocida capsule biosynthetic operon (e.g., phyB, phyA, hyaE, hyaD, hyaC, hyaB, hexD, hexC, hexB, and/or hexA).

The methods of the present invention are useful for inducing cross-protective immunity in a variety of animals including, e.g., birds, swine and cattle. In certain exemplary embodiments, the methods of the present invention are useful for inducing protective immunity in a poultry bird such as, e.g., a chicken or a turkey. Such methods are particularly useful for inducing protection against fowl cholera caused by virulent strains of P. multocida.

The invention includes single vaccination regimens as well as multiple vaccination regimens. In multiple vaccination regimens, a first dose of mutant P. multocida is administered to an animal at a first point in time, and then a second dose of mutant P. multocida is administered to the animal at a later point in time.

The foregoing and additional exemplary embodiments of the present invention are described in detail elsewhere herein.

### DETAILED DESCRIPTION OF THE INVENTION

It is known in the art that Pastuerella moltocida (P. multocida) isolates can be classified in terms of serogroup and serotype. The different serogroups of P. multocida include serogroups A, B, D, E and F. (Carter, Adv. Vet. Sci. 11:321-379 (1967)). The different serotypes include serotypes 1, 2, 3, 4, 3×4, 5, 6, 7, 8, 9, 10, 11, 12, 13, 14, 15, and 16. (Rimler and Rhodes, J. Clin. Microbiol. 25:616-618 (1987)). Particular strains of P. multocida are therefore commonly designated by both serogroup and serotype, in terms such as, e.g., “A:1,” “A:3,” “A:4,” “A:3×4,” etc. The convention of designating P. multocida by serogroup:serotype will be used herein, and the serotype:serogroup designation of a P. multocida isolate may sometimes be referred to simply as the “serotype” of that isolate.

The present invention is based, in part, on the surprising discovery that an attenuated strain of P. multocida having a particular serotype can induce protective immunity in an animal against a virulent strain of P. multocida having a different serotype. For example, it was discovered by the present inventors that an attenuated A:3 strain of P. multocida is capable of inducing protective immunity, not only against a virulent A:3 strain of P. multocida, but also against a virulent A:1 strain of P. multocida. Accordingly, the present invention provides methods for inducing cross-protective immunity against multiple serotypes of P. multocida.

In one embodiment, the invention provides methods for inducing cross-protective immunity against a virulent P. multocida strain. The methods comprise administering to an animal a mutant P. multocida strain. According to the present invention, the serotype of the virulent P. multocida strain (against which protective immunity is sought to be induced) is different from the serotype of the mutant P. multocida strain. The mutant P. multocida strain may induce protective immunity against multiple virulent P. multocida serotypes, at least one of which differs from the serotype of the mutant P. multocida strain itself. For example, in accordance with the present invention, an A:3 mutant P. multocida strain may, in certain embodiments, induce protective immunity against both an A:1 virulent P. multocida strain and an A:3 virulent P. multocida strain. Alternatively, an A:1 mutant P. multocida strain may, in certain other embodiments, induce protective immunity against both an A:3 virulent P. multocida strain and an A:1 virulent P. multocida strain.

### Virulent P. multocida

As used herein, the expression “virulent P. multocida strain” means a strain of P. multocida that, when administered to an animal, causes a disease in that animal. For example, a virulent P. multocida strain can be a strain that causes, e.g., fowl cholera in poultry, atrophic rhinitis in swine, and/or hemorrhagic septicemia in cattle.

### Mutant P. multocida

The mutant P. multocida strain used in the context of the present invention may be an acapsular mutant strain of P. multocida. As used herein, the term “acapsular” means that the P. multocida cells lack part or all of the extracellular polysaccharide-containing capsule. In specific embodiments, the acapsular P. multocida mutant strains lack the entire extracellular polysaccharide-containing capsule. The acapsular P. multocida mutant strain may be obtained by selecting random P. multocida mutants that lack the capsule. Acapsular P. multocida mutant strains may be obtained by repeated subculturing of capsular P. multocida cells. (Waft et al., FEMS Microbiol. Lett. 225:9-14 (2003)). Alternatively, acapsular P. multocida mutants may be obtained by enzymatic removal of the capsule.

The mutant P. multocida strains used in the context of the present invention may, in certain embodiments, contain one or more mutations that cause the mutant P. multocida to be acapsular. The mutant P. multocida strains used in the context of the present invention may, in certain other embodiments, contain one or more mutations that cause the mutant P. multocida to be attenuated. Preferably, the mutations that cause the mutant P. multocida to be acapsular also cause the mutant P. multocida to be attenuated.

According to the present invention, the mutations that cause the mutant P. multocida to be acapsular may be any mutation(s) that directly or indirectly affect the formation and/or maintenance of the capsule surrounding the P. multocida cells. For example, the mutations may impair or inhibit the expression of one or more genes found within the P. multocida capsule biosynthetic operon. (Watt et al., FEMS Microbiol. Lett. 225:9-14 (2003)). The genes found within the P. multocida capsule biosynthetic operon include phyB, phyA, hyaE, hyaD, hyaC, hyaB, hexD, hexC, hexB, and hexA. (Watt et al., FEMS Microbiol. Lett. 225:9-14 (2003)).

The mutation(s) can be inside or outside of the coding region of one or more genes found within the P. multocida capsule biosynthetic operon. For example, the mutation(s) may be within or near a promoter or enhancer that controls or regulates the expression of one or more capsule biosynthetic genes. Alternatively or additionally, the mutation(s) may be within the open reading frame (ORF) of one or more capsule biosynthetic genes. Exemplary mutations include insertions, deletions, and substitutions of one or more nucleotides. In certain embodiments, the mutation comprises a marker gene inserted into the ORF of one or more capsule biosynthetic genes. The marker gene may be inserted in place of nucleotides that are normally found in the ORF. The marker gene may be, e.g., a gene whose gene product confers antibiotic resistance to bacteria, or a gene that encodes a detectable gene product.

In certain embodiments of the present invention, the mutant P. multocida strains contain a deletion of all or part of the coding region of a capsule biosynthetic gene. Exemplary deletion mutations of the P. multocida hyaE gene which can be used in the context of the present invention include, e.g., any of the deletion mutations set forth in U.S. Patent Appl. Publication No. 2005/0106185. Exemplary mutations of the P. multocida hexA gene that can be used in the context of the present invention include, e.g., the hexA mutation set forth in Chung et al., Infect. Immun. 69:2487-2492 (2001).

According to the present invention, a mutant P. multocida strain is considered “attenuated” if the percentage of animals exhibiting one or more disease symptoms associated with P. multocida infection after receiving a particular dose of mutant P. multocida cells is less than the percentage of animals exhibiting one or more disease symptoms associated with P. multocida infection after receiving the same dose of wild-type P. multocida cells. For example, if 95% of animals receiving a particular dose of mutant P. multocida cells exhibit one or more disease symptoms associated with P. multocida infection, while 100% of animals receiving the same dose of wild-type P. multocida cells exhibit one or more disease symptoms associated with P. multocida infection, then the mutant P. multocida cells are deemed “attenuated.” Mutant P. multocida cells will be considered “attenuated” if 5%, 10%, 15%, 20%, 25%, 30%, 35%, 40%, 45%, 50%, 55%, 60%, 65%, 70%, 75%, 80%, 85%, 90%, 95%, or 100% fewer animals exhibit one or more disease symptoms associated with P. multocida infection after receiving a particular dose of the mutant P. multocida cells as compared to the percentage of animals exhibiting one or more disease symptoms associated with P. multocida infection after receiving the same dose of wild-type P. multocida cells.

Alternative methods for determining whether mutant P. multocida cells are attenuated are known in the art and can be used in the context of the present invention. For example, mutant P. multocida cells will be considered “attenuated” if the number of mutant cells required to kill half of a population of target animals (expressed as “LD₅₀”) is greater than the number of wild-type cells required to kill half of a population of susceptible target animals. Mutant P. multocida cells will also be considered “attenuated” if the extent, number and/or severity of pathological lesions in a population of animals exposed to a particular dose of mutant cells is less than the extent, number and/or severity of pathological lesions observed in a population of animals exposed to the same dose of wild-type cells.

### Cross-Protective Immunity

The methods of the present invention are useful for inducing cross-protective immunity in animals against virulent P. multocida strains. As used herein, the expression “cross-protective immunity” means that a mutant P. multocida strain, when administered to an animal, will induce “protective immunity” (defined hereinbelow) against at least one virulent P. multocida strain having a serotype that is different from the mutant P. multocida strain. The mutant P. multocida strain may, in certain embodiments, additionally induce protective immunity against a virulent strain of P. multocida having a serotype that is the same as the serotype of the mutant P. multocida. Non-limiting, examples of cross-protective immunity are illustrated in Table I:

TABLE I
ILLUSTRATIVE EXAMPLES OF CROSS-PROTECTIVE IMMUNITY

| A mutant | . . . induces protective immunity in an animal |
|---|---|
| P. multocida having | against virulent P. multocida having this/these |
| this serotype . . . | serotype(s) |
| A:1 | A:3 |
| A:1 | A:4 |
| A:1 | A:3 × 4 |
| A:1 | A:3 and A:4 |
| A:1 | A:3 and A:3 × 4 |
| A:1 | A:4 and A:3 × 4 |
| A:1 | A:3, A:4 and A:3 × 4 |
| A:1 | A:3 and A:1 |
| A:1 | A:4 and A:1 |
| A:1 | A:3 × 4 and A:1 |
| A:1 | A:3, A:4 and A:1 |
| A:1 | A:3, A:3 × 4 and A:1 |
| A:1 | A:4, A:3 × 4 and A:1 |
| A:1 | A:3, A:4, A:3 × 4 and A:1 |
| A:3 | A:1 |
| A:3 | A:4 |
| A:3 | A:3 × 4 |
| A:3 | A:1 and A:4 |
| A:3 | A:1 and A:3 × 4 |
| A:3 | A:4 and A:3 × 4 |
| A:3 | A:1, A:4 and A:3 × 4 |
| A:3 | A:1 and A:3 |
| A:3 | A:4 and A:3 |
| A:3 | A:3 × 4 and A:3 |
| A:3 | A:1, A:4 and A:3 |
| A:3 | A:1, A:3 × 4 and A:3 |
| A:3 | A:4, A:3 × 4 and A:3 |
| A:3 | A:1, A:4, A:3 × 4 and A:3 |

Additional examples of cross-protective immunity are within the scope of the present invention.

### Protective Immunity

As used herein, the expression “protective immunity” refers to an immune response in a host animal (either active/acquired or passive/innate, or both) which, leads to inactivation and/or reduction in the load of virulent P. multocida and to generation of long-lasting immunity (that is acquired, e.g., through production of antibodies), which prevents or delays the development of a disease upon repeated exposure to the same or a related virulent P. multocida strain. A “protective immune response” comprises a humoral (antibody) immunity or cellular immunity, or both, effective to, e.g., eliminate or reduce the load of virus or produce any other measurable alleviation of the infection. The phrase “induce an immune response,” within the meaning of the present invention, refers to the property or process of increasing the scale and/or efficiency of immunoreactivity to a virulent strain of P. multocida. In the context of the present invention, the immunoreactivity is preferably a cellular immunity, most preferably CD4+ and/or CD8+ T cell-mediated immunity. An immune response is believed to be induced, if any measurable parameter of antigen-specific immunoreactivity (e.g., T-cell production) is increased at least two-fold, preferably ten-fold, most preferably thirty-fold.

### Administration of Mutant P. multocida to Animals

The methods of the present invention are useful for inducing protective immunity in animals such as birds, and ungulates. “Birds” include wild (e.g., game fowl) and domesticated (e.g., poultry or pet) birds and includes both adult and developing forms (e.g., hatchlings, chicks, poults, etc.). “Poultry” or “poultry birds” include all birds kept, harvested, or domesticated for meat or eggs, including chicken, turkey, ostrich, game hen, squab, guinea fowl, pheasant, quail, duck, goose, and emu. “Ungulates” include, but are not limited to, cattle (bovine animals), water buffalo, bison, sheep, swine, deer, elephants, and yaks. Each of these includes both adult and developing forms (e.g., calves, piglets, lambs, etc.).

The mutant P. multocida can be administered by a variety of routes. The route of administration may depend on the type of animal to which the mutant P. multocida are administered.

For example, mutant P. multocida may be conveniently administered to ungulates by oral administration (e.g., in the feed or drinking water or in bait). It is particularly convenient to top-dress or mix feed with the mutant P. multocida. Other routes for vaccination can also be used with ungulates including, e.g., subcutaneous, intramuscular, intravenous, intradermal, intranasal, intrabronchial, etc. Mutant P. multocida of the invention can be implanted in the ear. Mutant P. multocida also can be administered by airspray, by eye inoculation, or by scarification.

If administered to birds, the mutant P. multocida of the invention may be conveniently administered by, e.g., mucosal or intramuscular injection. In a hatchery, mutant P. multocida of the invention can be administered using techniques such as, e.g., in ovo vaccination, spray vaccination, or subcutaneous vaccination. On the farm, mutant P. multocida of the invention can be administered to birds using techniques such as scarification, spray vaccination, eye drop vaccination, in-water vaccination, in-feed vaccination, wing web vaccination, subcutaneous vaccination, and intramuscular vaccination.

The amount of mutant P. multocida administered to the animals in the context of the present invention will vary based on the type and size of the animal and the route of administration. Large animals such as, e.g., livestock/ungulates may be administered between about 10⁶ to about 10⁹ cfu of mutant P. multocida per dose. For example, a dose of mutant P. multocida to be administered to a large ungulate (e.g., cattle, etc.) may contain about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸, about 1×10⁹, about 2×10⁹, about 3×10⁹, about 4×10⁹, about 5×10⁹, about 6×10⁹, about 7×10⁹, about 8×10⁹ or about 9×10⁹ cfu of mutant P. multocida. Smaller livestock/ungulates such as, e.g., sheep and swine, may be administered between about 10⁴ to about 10⁸ cfu of mutant P. multocida. For example, a dose of mutant P. multocida to be administered to a smaller ungulate may contain about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷ or about 9×10⁷ cfu of mutant P. multocida. Analogous dosing amounts can be readily deduced for companion animals.

For administration to birds, the amount of mutant P. multocida per dose can be, e.g., from about 10² to about 10⁸ cfu, depending on the size of the bird and route of administration. For example, a dose of mutant P. multocida to be administered to a bird may contain about 1×10², about 2×10², about 3×10², about 4×10², about 5×10², about 6×10², about 7×10², about 8×10², about 9×10², about 1×10³, about 2×10³, about 3×10³, about 4×10³, about 5×10³, about 6×10³, about 7×10³, about 8×10³, about 9×10³, about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10′, about 1×10³, about 2×10³, about 3×10⁸, about 4×10⁸, about 5×10⁸, about 6×10⁸, about 7×10⁸, about 8×10⁸, about 9×10⁸ cfu of mutant P. multocida.

The mutant P. multocida of the present invention may be administered to birds as a single vaccination or as multiple (e.g., 2, 3, 4, 5, 6, 7, 8, 9, 10 or more) vaccinations. The single or first vaccination may occur in ovo (prior to hatching), or at any time after hatching. For example, if in ovo administration is used, the single or first vaccination may occur on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25 or on day 26 of incubation (the first day of incubation is considered day 0; eggs are laid approximately 2-7 days before incubation). If mutant P. multocida are administered after hatching, the single or first vaccination may occur on day 1, day 2, day 3, day 4, day 5, day 6, day 7, day 8, day 9 or day 10 post hatch, or at 1, 2, 3, 4, 5, 6, 7, 8, 9, 10, 11, 12, 13, 14, 15, 16, 17, 18, 19, or 20 weeks of age.

Exemplary dosing regimens within the scope of the present invention are set forth in Table II.

TABLE II

| Vaccination 1 | | | Vaccination 2 | |
|---|---|---|---|---|
| | | Amount of | | Amount of |
| | | mutant P. multocida/ | | mutant P. multocida/ |
| Exemplary | Time of | dose | Time of | dose |
| Regimen # | vaccination | (CFU) | vaccination | (CFU) |
| 1 | 4 weeks old | 1.1 × 10⁶-1.6 × 10⁶ | 5 weeks old | 4.2 × 10⁵-2.2 × 10⁷ |
| 2 | 4 weeks old | 1.1 × 10⁶-1.6 × 10⁶ | 6 weeks old | 4.2 × 10⁵-2.2 × 10⁷ |
| 3 | 4 weeks old | 1.1 × 10⁶-1.6 × 10⁶ | 7 weeks old | 4.2 × 10⁵-2.2 × 10⁷ |
| 4 | 4 weeks old | 1.1 × 10⁶-1.6 × 10⁶ | 8 weeks old | 4.2 × 10⁵-2.2 × 10⁷ |
| 5 | 4 weeks old | 1.1 × 10⁶-1.6 × 10⁶ | 9 weeks old | 4.2 × 10⁵-2.2 × 10⁷ |
| 6 | 4 weeks old | 1.1 × 10⁶-1.6 × 10⁶ | 10 weeks old | 4.2 × 10⁵-2.2 × 10⁷ |
| 7 | 4 weeks old | 1.1 × 10⁶-1.6 × 10⁶ | 11 weeks old | 4.2 × 10⁵-2.2 × 10⁷ |
| 8 | 4 weeks old | 1.1 × 10⁶-1.6 × 10⁶ | 12 weeks old | 4.2 × 10⁵-2.2 × 10⁷ |
| 9 | 4 weeks old | 1.1 × 10⁶-1.6 × 10⁶ | 13 weeks old | 4.2 × 10⁵-2.2 × 10⁷ |
| 10 | 4 weeks old | 1.1 × 10⁶-1.6 × 10⁶ | 14 weeks old | 4.2 × 10⁵-2.2 × 10⁷ |
| 11 | 5 weeks old | 1.1 × 10⁶-1.6 × 10⁶ | 6 weeks old | 4.2 × 10⁵-2.2 × 10⁷ |
| 12 | 5 weeks old | 1.1 × 10⁶-1.6 × 10⁶ | 7 weeks old | 4.2 × 10⁵-2.2 × 10⁷ |
| 13 | 5 weeks old | 1.1 × 10⁶-1.6 × 10⁶ | 8 weeks old | 4.2 × 10⁵-2.2 × 10⁷ |
| 14 | 5 weeks old | 1.1 × 10⁶-1.6 × 10⁶ | 9 weeks old | 4.2 × 10⁵-2.2 × 10⁷ |
| 15 | 5 weeks old | 1.1 × 10⁶-1.6 × 10⁶ | 10 weeks old | 4.2 × 10⁵-2.2 × 10⁷ |
| 16 | 5 weeks old | 1.1 × 10⁶-1.6 × 10⁶ | 11 weeks old | 4.2 × 10⁵-2.2 × 10⁷ |
| 17 | 5 weeks old | 1.1 × 10⁶-1.6 × 10⁶ | 12 weeks old | 4.2 × 10⁵-2.2 × 10⁷ |
| 18 | 5 weeks old | 1.1 × 10⁶-1.6 × 10⁶ | 13 weeks old | 4.2 × 10⁵-2.2 × 10⁷ |
| 19 | 5 weeks old | 1.1 × 10⁶-1.6 × 10⁶ | 14 weeks old | 4.2 × 10⁵-2.2 × 10⁷ |
| 20 | 5 weeks old | 1.1 × 10⁶-1.6 × 10⁶ | 15 weeks old | 4.2 × 10⁵-2.2 × 10⁷ |
| 21 | 6 weeks old | 1.1 × 10⁶-1.6 × 10⁶ | 7 weeks old | 4.2 × 10⁵-2.2 × 10⁷ |
| 22 | 6 weeks old | 1.1 × 10⁶-1.6 × 10⁶ | 8 weeks old | 4.2 × 10⁵-2.2 × 10⁷ |
| 23 | 6 weeks old | 1.1 × 10⁶-1.6 × 10⁶ | 9 weeks old | 4.2 × 10⁵-2.2 × 10⁷ |
| 24 | 6 weeks old | 1.1 × 10⁶-1.6 × 10⁶ | 10 weeks old | 4.2 × 10⁵-2.2 × 10⁷ |
| 25 | 6 weeks old | 1.1 × 10⁶-1.6 × 10⁶ | 11 weeks old | 4.2 × 10⁵-2.2 × 10⁷ |
| 26 | 6 weeks old | 1.1 × 10⁶-1.6 × 10⁶ | 12 weeks old | 4.2 × 10⁵-2.2 × 10⁷ |
| 27 | 6 weeks old | 1.1 × 10⁶-1.6 × 10⁶ | 13 weeks old | 4.2 × 10⁵-2.2 × 10⁷ |
| 28 | 6 weeks old | 1.1 × 10⁶-1.6 × 10⁶ | 14 weeks old | 4.2 × 10⁵-2.2 × 10⁷ |
| 29 | 6 weeks old | 1.1 × 10⁶-1.6 × 10⁶ | 15 weeks old | 4.2 × 10⁵-2.2 × 10⁷ |
| 30 | 6 weeks old | 1.1 × 10⁶-1.6 × 10⁶ | 16 weeks old | 4.2 × 10⁵-2.2 × 10⁷ |
| 31 | 7 weeks old | 1.1 × 10⁶-1.6 × 10⁶ | 8 weeks old | 4.2 × 10⁵-2.2 × 10⁷ |
| 32 | 7 weeks old | 1.1 × 10⁶-1.6 × 10⁶ | 9 weeks old | 4.2 × 10⁵-2.2 × 10⁷ |
| 33 | 7 weeks old | 1.1 × 10⁶-1.6 × 10⁶ | 10 weeks old | 4.2 × 10⁵-2.2 × 10⁷ |
| 34 | 7 weeks old | 1.1 × 10⁶-1.6 × 10⁶ | 11 weeks old | 4.2 × 10⁵-2.2 × 10⁷ |
| 35 | 7 weeks old | 1.1 × 10⁶-1.6 × 10⁶ | 12 weeks old | 4.2 × 10⁵-2.2 × 10⁷ |
| 36 | 7 weeks old | 1.1 × 10⁶-1.6 × 10⁶ | 13 weeks old | 4.2 × 10⁵-2.2 × 10⁷ |
| 37 | 7 weeks old | 1.1 × 10⁶-1.6 × 10⁶ | 14 weeks old | 4.2 × 10⁵-2.2 × 10⁷ |
| 38 | 7 weeks old | 1.1 × 10⁶-1.6 × 10⁶ | 15 weeks old | 4.2 × 10⁵-2.2 × 10⁷ |
| 39 | 7 weeks old | 1.1 × 10⁶-1.6 × 10⁶ | 16 weeks old | 4.2 × 10⁵-2.2 × 10⁷ |
| 40 | 7 weeks old | 1.1 × 10⁶-1.6 × 10⁶ | 17 weeks old | 4.2 × 10⁵-2.2 × 10⁷ |
| 41 | 8 weeks old | 1.1 × 10⁶-1.6 × 10⁶ | 9 weeks old | 4.2 × 10⁵-2.2 × 10⁷ |
| 42 | 8 weeks old | 1.1 × 10⁶-1.6 × 10⁶ | 10 weeks old | 4.2 × 10⁵-2.2 × 10⁷ |
| 43 | 8 weeks old | 1.1 × 10⁶-1.6 × 10⁶ | 11 weeks old | 4.2 × 10⁵-2.2 × 10⁷ |
| 44 | 8 weeks old | 1.1 × 10⁶-1.6 × 10⁶ | 12 weeks old | 4.2 × 10⁵-2.2 × 10⁷ |
| 45 | 8 weeks old | 1.1 × 10⁶-1.6 × 10⁶ | 13 weeks old | 4.2 × 10⁵-2.2 × 10⁷ |
| 46 | 8 weeks old | 1.1 × 10⁶-1.6 × 10⁶ | 14 weeks old | 4.2 × 10⁵-2.2 × 10⁷ |
| 47 | 8 weeks old | 1.1 × 10⁶-1.6 × 10⁶ | 15 weeks old | 4.2 × 10⁵-2.2 × 10⁷ |
| 48 | 8 weeks old | 1.1 × 10⁶-1.6 × 10⁶ | 16 weeks old | 4.2 × 10⁵-2.2 × 10⁷ |
| 49 | 8 weeks old | 1.1 × 10⁶-1.6 × 10⁶ | 17 weeks old | 4.2 × 10⁵-2.2 × 10⁷ |
| 50 | 8 weeks old | 1.1 × 10⁶-1.6 × 10⁶ | 18 weeks old | 4.2 × 10⁵-2.2 × 10⁷ |
| 51 | 9 weeks old | 1.1 × 10⁶-1.6 × 10⁶ | 10 weeks old | 4.2 × 10⁵-2.2 × 10⁷ |
| 52 | 9 weeks old | 1.1 × 10⁶-1.6 × 10⁶ | 11 weeks old | 4.2 × 10⁵-2.2 × 10⁷ |
| 53 | 9 weeks old | 1.1 × 10⁶-1.6 × 10⁶ | 12 weeks old | 4.2 × 10⁵-2.2 × 10⁷ |
| 54 | 9 weeks old | 1.1 × 10⁶-1.6 × 10⁶ | 13 weeks old | 4.2 × 10⁵-2.2 × 10⁷ |
| 55 | 9 weeks old | 1.1 × 10⁶-1.6 × 10⁶ | 14 weeks old | 4.2 × 10⁵-2.2 × 10⁷ |
| 56 | 9 weeks old | 1.1 × 10⁶-1.6 × 10⁶ | 15 weeks old | 4.2 × 10⁵-2.2 × 10⁷ |
| 57 | 9 weeks old | 1.1 × 10⁶-1.6 × 10⁶ | 16 weeks old | 4.2 × 10⁵-2.2 × 10⁷ |
| 58 | 9 weeks old | 1.1 × 10⁶-1.6 × 10⁶ | 17 weeks old | 4.2 × 10⁵-2.2 × 10⁷ |
| 59 | 9 weeks old | 1.1 × 10⁶-1.6 × 10⁶ | 18 weeks old | 4.2 × 10⁵-2.2 × 10⁷ |
| 60 | 9 weeks old | 1.1 × 10⁶-1.6 × 10⁶ | 19 weeks old | 4.2 × 10⁵-2.2 × 10⁷ |
| 61 | 10 weeks old | 1.1 × 10⁶-1.6 × 10⁶ | 11 weeks old | 4.2 × 10⁵-2.2 × 10⁷ |
| 62 | 10 weeks old | 1.1 × 10⁶-1.6 × 10⁶ | 12 weeks old | 4.2 × 10⁵-2.2 × 10⁷ |
| 63 | 10 weeks old | 1.1 × 10⁶-1.6 × 10⁶ | 13 weeks old | 4.2 × 10⁵-2.2 × 10⁷ |
| 64 | 10 weeks old | 1.1 × 10⁶-1.6 × 10⁶ | 14 weeks old | 4.2 × 10⁵-2.2 × 10⁷ |
| 65 | 10 weeks old | 1.1 × 10⁶-1.6 × 10⁶ | 15 weeks old | 4.2 × 10⁵-2.2 × 10⁷ |
| 66 | 10 weeks old | 1.1 × 10⁶-1.6 × 10⁶ | 16 weeks old | 4.2 × 10⁵-2.2 × 10⁷ |
| 67 | 10 weeks old | 1.1 × 10⁶-1.6 × 10⁶ | 17 weeks old | 4.2 × 10⁵-2.2 × 10⁷ |
| 68 | 10 weeks old | 1.1 × 10⁶-1.6 × 10⁶ | 18 weeks old | 4.2 × 10⁵-2.2 × 10⁷ |
| 69 | 10 weeks old | 1.1 × 10⁶-1.6 × 10⁶ | 19 weeks old | 4.2 × 10⁵-2.2 × 10⁷ |
| 70 | 10 weeks old | 1.1 × 10⁶-1.6 × 10⁶ | 20 weeks old | 4.2 × 10⁵-2.2 × 10⁷ |

Additional dosing regimens, besides those set forth in Table II, are included within the scope of the present invention.

Vaccines comprising mutant P. multocida can be given alone or as a component of a polyvalent vaccine, e.g., in combination with other vaccines. Mutant P. multocida in a vaccine formulation can be live or killed; either live or killed bacteria can be lyophilized and, optionally, reconstituted as is known in the art. Vaccines can conveniently be provided in kits, which also can comprise appropriate labeling and instructions for administering a vaccine to an animal subject (e.g., livestock, an ungulate, a companion animal) or a bird (e.g., poultry).

Vaccines comprising mutant P. multocida also can comprise pharmaceutically and veterinarily acceptable carriers. Such carriers are well known to those in the art and include, but are not limited to, large, slowly metabolized macromolecules, such as proteins, polysaccharides, polylactic acids, polyglycolic acids, polymeric amino acids, amino acid copolymers, and inactive virus particles. Pharmaceutically and veterinarily acceptable salts can also be used in the vaccine, for example, mineral salts such as hydrochlorides, hydrobromides, phosphates, or sulfates, as well as the salts of organic acids such as acetates, proprionates, malonates, or benzoates. Vaccines also can contain liquids, such as water, saline, glycerol, and ethanol, as well as substances such as wetting agents, emulsifying agents, or pH buffering agents. Liposomes also can be used as carriers for mutant bacteria. See U.S. Pat. No. 5,422,120, WO 95/13796, WO 91/14445, or EP 524,968 B1.

If desired, an adjuvant can be added to a vaccine. Useful adjuvants include, without limitation, surfactants (e.g., hexadecylamine, octadecylanine, lysolecithin, dimethyldioctadecylammonium bromide, N,N-dioctadecyl-n′-N-bis(2-hydroxyethylpropane di-amine), methoxyhexadecylglycerol, and pluronic polyols); polyanions (e.g., pyran, dextran sulfate, poly IC, polyacrylicacid, carbopol), peptides (e.g., muramyl dipeptide, dimethylglycine, tuftsin), oil emulsions, alum, and mixtures thereof.

The following examples are illustrative, but not limiting, of the method and compositions of the present invention. Other suitable modifications and adaptations of the variety of conditions and parameters normally encountered in molecular biology, virology, immunology and chemistry which are obvious to those skilled in the art in view of the present disclosure are within the spirit and scope of the invention.

### EXAMPLES

### Example 1

### Protection of Turkeys Against a Virulent A:3 Strain of P. multocida Using a hyaE Deletion Mutant A:3 Strain of P. multocida

### Introduction

A preliminary safety study has shown that a P. multocida hyaE mutant A:3 strain, PM1059hyaE (see U.S. Patent Appl. Publication No. 2005/0106185) is safe in chickens when administered by oral gavage, intramuscular injection, intratracheal inoculation, drinking water and wing-web. No mortality was observed in any of the chickens administered with this strain by any of the five routes. The inoculation did not have significant effects on body weight of the chickens.

In this Example, the efficacy of PM1059hyaE in turkeys against challenge with a virulent A:3 strain of P. multocida is assessed.

### Materials and Methods

### Event Log

TABLE III

| Bird Age | Procedure |
|---|---|
| 6 weeks old | Pre-vaccination bleeding and first vaccination |
| 10 weeks old | Second vaccination |
| 13 weeks old | Pre-challenge bleeding and challenge |
| 15 weeks old | Mortality observation |

### Animal Selection

Thirty commercial turkeys are used in the study. The turkeys are of mixed sex and are wing-banded prior to the start of the study.

All turkeys are housed in floor-pens (one room for each group) until the completion of the study. All turkeys are under veterinary care and are fed with a standard antibiotic-free commercial feed, with feed and water available ad libitum.

### Vaccine

The vaccine contains P. multocida hyaE mutant strain PM1059hyaE (see U.S. Patent Appl. Publication No. 2005/0106185) at the passage level of X+5. The vaccine is stored in lyophilized vials at 4° C. to 8° C.

### Experimental Design

TABLE IV

| Test | | Targeted Inoculation | | No. |
|---|---|---|---|---|
| Group | Treatment | (CFU per bird) | Inoculation | Birds |
| 1 | PM1059hyaE | 1.0 × 10⁷ CFU/0.5 | Intramuscular | 20 |
| | | mL dose | | |
| 2 | None | N/A | N/A | 10 |

### RANDOMIZATION

Birds are placed randomly into two groups at the time of the first vaccination according to random numbers generated with a commercially available computer spreadsheet program. At the time of the first vaccination, birds are wing-banded and blood samples are collected. Birds in Test Groups 1 and 2 are housed in separate floor pens.

### Vaccination

For Test Group 1, the vaccine (A:3 mutant strain PM1059hyaE) is administered by intramuscular injection into breast muscle with a target titer of 1.0×10⁷ CFU in 0.5 mL PBS for each bird.

For Test Group 2, 0.5 mL of PBS is administered to the birds. These birds serve as challenge controls.

### Post-Inoculation Observation

Following each vaccination, all birds are observed daily for general health and mortality until challenge.

### Challenge Procedure and Post-Challenge Observation

Three weeks after the second vaccination, all birds in both groups are challenged with a virulent A:3 strain of P. multocida at a target titer of 1.0×10⁸ CFU per 0.2 mL per bird. The challenge strain is administered by intramuscular injection into the breast muscle. The challenged birds are observed daily for 14 days for mortality.

### Results

The percent of birds from Groups 1 and 2 that died following challenge with virulent A:3 P. multocida strain 1059 is summarized in Table V.

TABLE V

Mortality (Post-Challenge)

| | No. Dead/Total No. | |
|---|---|---|
| | Birds | Percent |
| Group 1 (PM1059hyaE) | 0/16 | 0% |
| Group 2 (Control) | 10 | 100% |

### Conclusion

This Example confirms that an A:3 acapsular mutant strain of P. multocida having a deletion of the hyaE gene, when administered to turkeys, provides protection against a virulent A:3 challenge strain of P. multocida.

### Example 2

### An A:3 P. multocida hyaE Deletion Mutant Induces Cross-Protective Immunity Against a Virulent A:1 Strain of P. multocida in Chickens

### Introduction

Generally, wild-type A:3 strains of P. multocida are virulent in turkeys but not in chickens. Therefore, an attenuated A:3 strain of P. multocida would not be expected to induce cross-protective immunity against a virulent A:1 P. multocida strain in chickens. In this Example, however, the safety and efficacy of P. multocida hyaE mutant A:3 strain, PM1059hyaE (see U.S. Patent Appl. Publication No. 2005/0106185) in chickens against challenge with a virulent A:1 strain of P. multocida is shown.

### Materials and Methods

### Event Log

TABLE VI

| Bird Age | Procedure |
|---|---|
| 1 day of age | Hatching |
| 8 weeks old | First vaccination |
| 12 weeks old | Second vaccination |
| 15 weeks old | Challenge |
| 17 weeks old | Mortality observation |

### Animal Selection

One hundred specific pathogen free (SPF) white leghorn chickens are used in the study. The chickens are of mixed sex and are wing-banded prior to the start of the study.

All chickens are housed in floor-pens (one room for each group) until the completion of the study. All chickens are under veterinary care and are fed a standard antibiotic-free commercial feed, with feed and water available ad libitum.

### Vaccine

The vaccine contains P. multocida HyaE mutant strain PM1059hyaE (see U.S. Patent Appl. Publication No. 2005/0106185) at the passage level of X+5. The vaccine is stored in lyophilized vials at 4° C. to 8° C.

### Experimental Design

TABLE VII

| Test | | Targeted Inoculation | | No. |
|---|---|---|---|---|
| Group | Treatment | (CFU per bird) | Inoculation | Birds |
| 1 | PM1059hyaE | 1.0 × 10⁷ CFU/ | Wing-web | 20 |
| | | 10 μL dose | | |
| 2 | PM1059hyaE | 1.0 × 10⁷ CFU/ | Oral gavage | 20 |
| | | 1 mL dose | | |
| 3 | PM1059hyaE | 1.0 × 10⁷ CFU/ | Intramuscular | 20 |
| | | 0.5 mL dose | | |
| 4 | CHOLERVAC- | Manufacturer's | Wing-web | 20 |
| | PM-1 ™ | instructions | | |
| 5 | None | N/A | N/A | 20 |

### Randomization

Birds are randomly placed into five groups at the time of the first vaccination according to random numbers generated with a commercially available computer spreadsheet program. At the time of the first vaccination, birds are wing-banded.

### Vaccination

For Test Group 1, the vaccine (A:3 mutant strain PM1059hyaE) is administered by wing-web at a titer of 1.19×10⁶ CFU/dose for the first vaccination and 4.48×10⁵ CFU/dose for the second vaccination for each bird.

For Test Group 2, the vaccine (A:3 mutant strain PM1059hyaE) is administered by oral gavage at a titer of 1.35×10⁶ CFU/dose for the first vaccination and 1.86×10⁷ CFU/dose for the second vaccination for each bird.

For Test Group 3, the vaccine (A:3 mutant strain PM1059hyaE) is administered by intramuscular injection into the breast muscle at a titer of 1.35×10⁶ CFU/dose for the first vaccination and 1.86×10⁷ CFU/dose for the second vaccination for each bird.

For Test Group 4, the vaccine (CHOLERVAC-PM-1™) was prepared and administered by wing-web according to the instructions of the manufacturer (Intervet, Whitby, Ontario, Canada).

Test Group 5 birds receive no treatment and serve as challenge controls.

All birds were weighed before the first vaccination and were weighed again before challenge.

### Post-Inoculation Observation

Following each vaccination, all birds are observed daily for general health and mortality until challenge.

### Challenge Procedure and Post-Challenge Observation

Three weeks after the second vaccination, all birds in all five groups are challenged with a virulent A:1 strain of P. multocida (strain X-73, Rimler, J. Clin. Microbiol 28:654-659 (1990)) at a titer of 1.53×10⁸ CFU per 0.2 mL per bird by intramuscular injection into breast muscle. The challenged birds are observed daily for 14 days for mortality.

### Results and Conclusion

The birds were observed for post-vaccination reactions or signs of Pasteurellosis. There were no signs of reactions to the vaccine and no signs of Pasteurellosis. There was one death in the intramuscular injection group (Group 3). The bird was necropsied and found to have died from abdominal bleeding, not attributed to the vaccine.

Birds were weighed before the first vaccination and before challenge in order to determine if the vaccine had any detrimental effect on weight gain. There was no significant difference in weights amongst the groups.

Results showing the survival percentage following challenge with virulent P. multocida for each of the vaccinated groups and the control group are summarized in Table VIII. As shown in Table VIII, intramuscular injection of a P. multocida hyaE mutant (A:3 mutant strain PM1059hyaE; Group 3) showed 100% protection against virulent A: 1 P. multocida (strain X-73).

TABLE VIII

| | No. Birds Challenged with 1.53 × 10⁸ CFU of virulent A:1 strain X-73 | | | | |
|---|---|---|---|---|---|
| | 20 | 19 | 20 | 20 | |
| Days | Group 1 | Group 2 | Group 3 | Group 4 | 20 |
| Post- | (Wing-Web) | (Oral Gavage) | (IM) | (CHLORVAC- | Group 5 |
| Challenge | PM1059hyaE | PM1059hyaE | PM1059hyaE | PM-1) | (Control) |
| 1 | 17 | 11 | 20 | 20 | 14 |
| 2 | 12 | 7 | 20 | 20 | 12 |
| 3 | 10 | 7 | 20 | 20 | 10 |
| 4 | 7 | 7 | 20 | 20 | 10 |
| 5 | 5 | 6 | 20 | 20 | 10 |
| 6 | 3 | 6 | 20 | 20 | 10 |
| 7 | 3 | 6 | 20 | 20 | 9 |
| 8 | 3 | 5 | 20 | 20 | 8 |
| 9 | 3 | 5 | 20 | 20 | 7 |
| 10 | 3 | 5 | 20 | 20 | 7 |
| 11 | 3 | 3 | 20 | 20 | 6 |
| 12 | 3 | 3 | 20 | 20 | 6 |
| 13 | 3 | 2 | 20 | 20 | 6 |
| 14 | 3 | 2 | 20 | 20 | 6 |
| | Percent Survival | | | | |
| | 15% | 10% | 100% | 100% | 30% |

The ability of an A:3 vaccine strain to cross-protect against a virulent A:1 challenge strain in chickens is unexpected. These results show that vaccination of animals with an acapsular mutant of P. multocida can provide broad cross-protection against virulent P. multocida strains having heterologous serotypes.

### Example 3

### Induction of Cross-Protective Immunity Against a Virulent A:3 Strain of P. multocida in Turkeys Using an Attenuated A:1 Strain of P. multocida

As noted in Example 2, wild-type A:3 strains of P. multocida are known to be virulent in turkeys but not in chickens. Conversely, wild-type A:1 strains of P. multocida are known to be virulent in chickens but not in turkeys. Thus, it is unexpected that an attenuated A:1 strain of P. multocida would induce cross-protective immunity against a virulent A:3 strain of P. multocida in turkeys.

To illustrate the cross-protective immunity induced by an attenuated A:1 strain of P. multocida in turkeys, an acapsular mutant of an A:1 P. multocida strain, such as strain X-73 (Rimler, J. Clin. Microbiol. 28:654-659 (1990)), is first obtained. The acapsular A:1 mutant includes a deletion of a gene within the P. multocida capsule biosynthetic operon, such as hexA. (Chung et al., Infect. Immun. 69:2487-2492 (2001)).

Two sets of commercial turkeys are used in this study. Each set contains equal numbers of turkeys. The first set is the Experimental set of turkeys which are administered about 1.0×10⁷ CFU of attenuated A:1 hexA mutant P. multocida at approximately 6 weeks of age. The second set is a Control set of turkeys that are administered buffer only at approximately 6 weeks of age.

At approximately 13 weeks of age, the turkeys in each set are challenged with about 1.0×10⁸ CFU of virulent A:3 strain of P. multocida. The turkeys in both the Experimental and Control groups are monitored for signs of P. multocida infection and/or mortality for at least 14 days after the challenge.

Cross-protective immunity is observed if fewer turkeys in the Experimental group exhibit signs of P. multocida infection at 14 days post-challenge. In other words, cross-protective immunity is observed if fewer turkeys that receive an attenuated, acapsular A:1 mutant strain of P. multocida, followed by challenge with a virulent A:3 strain of P. multocida, exhibit signs of P. multocida infection as compared to turkeys that do not receive the attenuated mutant A:1 strain prior to virulent challenge.

Although the foregoing invention has been described in some detail by way of illustration and example for purposes of clarity of understanding, this invention is not limited to the particular embodiments disclosed, but is intended to cover all changes and modifications that are within the spirit and scope of the invention as defined by the appended claims.

All publications and patents mentioned in this specification are indicative of the level of skill of those skilled in the art to which this invention pertains. All publications and patents are herein incorporated by reference to the same extent as if each individual publication or patent application were specifically and individually indicated to be incorporated by reference.

What is claimed is:

1. A method for inducing cross-protective immunity against virulent P. multocida, said method comprising administering to an animal a mutant P. multocida;
wherein said mutant P. multocida contains one or more mutations that cause said mutant P. multocida to be acapsular, and wherein said one or more mutations cause said mutant P. multocida to be attenuated;
wherein said mutant P. multocida induces protective immunity in said animal against said virulent P. multocida; and
wherein said virulent P. multocida has a serotype that is different from the serotype of said mutant P. multocida.

2. The method of claim 1, wherein said mutant P. multocida additionally induces protective immunity in said animal against a virulent P. multocida having a serotype that is the same as the serotype of said mutant P. multocida.

3. The method of claim 1, wherein said mutant P. multocida has a serotype selected from the group consisting of: A:1, A:3, A:4, and A:3×4.

4. The method of claim 3, wherein said mutant P. multocida is an A:1 P. multocida strain.

5. The method of claim 4, wherein said virulent P. multocida is an A:3, A:4 or A:3×4 P. multocida strain.

6. The method of claim 5, wherein said mutant A:1 P. multocida strain additionally induces protective immunity in said animal against a virulent A: 1 P. multocida strain.

7. The method of claim 4, wherein said mutant A:1 P. multocida strain induces protective immunity in said animal against virulent A:3, A:4 and A:3×4 P. multocida strains.

8. The method of claim 7, wherein said mutant A:1 P. multocida strain induces protective immunity in said animal against virulent A:1, A:3, A:4 and A:3×4 P. multocida strains.

9. The method of claim 3, wherein said mutant P. multocida is an A:3 P. multocida strain.

10. The method of claim 9, wherein said virulent P. multocida is an A:1, A:4 or A:3×4 P. multocida strain.

11. The method of claim 10, wherein said mutant A:3 P. multocida strain additionally induces protective immunity in said animal against a virulent A:3 P. multocida strain.

12. The method of claim 9, wherein said mutant A:3 P. multocida strain induces protective immunity in said animal against virulent A:1, A:4 and A:3×4 P. multocida strains.

13. The method of claim 12, wherein said mutant A:3 P. multocida strain induces protective immunity in said animal against virulent A:1, A:3, A:4 and A:3×4 P. multocida strains.

14. The method of claim 1, wherein said animal is a bird.

15. The method of claim 1, wherein said animal is an ungulate.

16. The method of claim 14, wherein said bird is a poultry bird.

17. The method of claim 16, wherein said poultry bird is a chicken, turkey, ostrich, game hen, squab, guinea fowl, pheasant, quail, duck, goose, or emu.

18. The method of claim 16, wherein said poultry bird is a chicken.

19. The method of claim 18, wherein said mutant P. multocida is an A:3 P. multocida strain.

20. The method of claim 2, wherein said poultry bird is a turkey.

21. The method of claim 20, wherein said mutant P. multocida is an A:1 P. multocida strain.

22. The method of claim 1, wherein said one or more mutations is a mutation that impairs the expression of one or more genes in the P. multocida capsule biosynthetic operon.

23. The method of claim 8, wherein said one or more genes in the P. multocida capsule biosynthetic operon is a gene selected from the group consisting of phyB, phyA, hyaE, hyaD, hyaC, hyaB, hexD, hexC, hexB, and hexA.

24. The method of claim 8, wherein said one or more mutations is an insertion, substitution, or a deletion mutation within the coding sequence of a gene selected from the group consisting of phyB, phyA, hyaE, hyaD, hyaC, hyaB, hexD, hexC, hexB, and hexA.

25. The method of claim 8, wherein said one or more mutations is a deletion of all or part of the coding sequence of a gene selected from the group consisting of phyB, phyA, hyaE, hyaD, hyaC, hyaB, hexD, hexC, hexB, and hexA.

26. The method of claim 11, wherein said one or more mutations is a deletion of all or part of the coding sequence of the hyaE gene.

27. A method for inducing cross-protective immunity in a chicken against a virulent A:1 P. multocida strain, said method comprising administering to said chicken an attenuated acapsular mutant A:3 P. multocida strain.

28. The method of claim 27, wherein said attenuated acapsular mutant A:3 P. multocida strain comprises a hyaE mutation in its genome.

29. A method for inducing cross-protective immunity in a turkey against a virulent A:3 P. multocida strain, said method comprising administering to said turkey an attenuated acapsular mutant A:1 P. multocida strain.

30. The method of claim 29, wherein said attenuated acapsular mutant A:1 P. multocida strain comprises a hyaE mutation in its genome.

31. A method for inducing cross-protective immunity against a virulent P. multocida, said method comprising administering to an animal a first dose of mutant P. multocida at a first time point, followed by administering to said animal a second dose of said mutant P. multocida at a second time point,
wherein said mutant P. multocida contains one or more mutations that cause said mutant P. multocida to be acapsular, and wherein said one or more mutations cause said mutant P. multocida to be attenuated;
wherein said mutant P. multocida induces protective immunity in said animal against said virulent P. multocida; and
wherein said virulent P. multocida has a serotype that is different from the serotype of said mutant P. multocida.

32. The method of claim 31, wherein said mutant P. multocida additionally induces protective immunity in said animal against a virulent P. multocida having a serotype that is the same as the serotype of said mutant P. multocida.

33. The method of claim 31, wherein said mutant P. multocida has a serotype selected from the group consisting of A:1, A:3, A:4, and A:3×4.

34. The method of claim 33, wherein said mutant P. multocida is an A:1 P. multocida strain.

35. The method of claim 34, wherein said virulent P. multocida is an A:3, A:4 or A:3×4 P. multocida strain.

36. The method of claim 35, wherein said mutant A:1 P. multocida strain additionally induces protective immunity in said animal against a virulent A:1 P. multocida strain.

37. The method of claim 34, wherein said mutant A:1 P. multocida strain induces protective immunity in said animal against virulent A:3, A:4 and A:3×4 P. multocida strains.

38. The method of claim 37, wherein said mutant A:1 P. multocida strain induces protective immunity in said animal against virulent A:1, A:3, A:4 and A:3×4 P. multocida strains.

39. The method of claim 33, wherein said mutant P. multocida is an A:3 P. multocida strain.

40. The method of claim 39, wherein said virulent P. multocida is an A:1, A:4 or A:3×4 P. multocida strain.

41. The method of claim 40, wherein said mutant A:3 P. multocida strain additionally induces protective immunity in said animal against a virulent A:3 P. multocida strain.

42. The method of claim 39, wherein said mutant A:3 P. multocida strain induces protective immunity in said animal against virulent A:1, A:4 and A:3×4 P. multocida strains.

43. The method of claim 42, wherein said mutant A:3 P. multocida strain induces protective immunity in said animal against virulent A:1, A:3, A:4 and A:3×4 P. multocida strains.

44. The method of claim 31, wherein said animal is a bird.

45. The method of claim 31, wherein said animal is an ungulate.

46. The method of claim 44, wherein said bird is a poultry bird.

47. The method of claim 46, wherein said poultry bird is a chicken, turkey, ostrich, game hen, squab, guinea fowl, pheasant, quail, duck, goose, or emu.

48. The method of claim 46, wherein said poultry bird is a chicken.

49. The method of claim 46, wherein said poultry bird is a turkey.

50. The method of claim 31, wherein said one or more mutations is a mutation that impairs the expression of one or more genes in the P. multocida capsule biosynthetic operon.

51. The method of claim 50, wherein said one or more genes in the P. multocida capsule biosynthetic operon is a gene selected from the group consisting of phyB, phyA, hyaE, hyaD, hyaC, hyaB, hexD, hexC, hexB, and hexA.

52. The method of claim 50, wherein said one or more mutations is an insertion, substitution, or a deletion mutation within the coding sequence of a gene selected from the group consisting of phyB, phyA, hyaE, hyaD, hyaC, hyaB, hexD, hexC, hexB, and hexA.

53. The method of claim 50, wherein said one or more mutations is a deletion of all or part of the coding sequence of a gene selected from the group consisting of phyB, phyA, hyaE, hyaD, hyaC, hyaB, hexD, hexC, hexB, and hexA.

54. The method of claim 53, wherein said one or more mutations is a deletion of all or part of the coding sequence of the hyaE gene.

55. The method of claim 31, wherein said first dose comprises from about 1×10² to about 1×10⁸ cfu of mutant P. multocida.

56. The method of claim 55, wherein said first dose comprises from about 1×10⁴ to about 1×10⁶ cfu of mutant P. multocida.

57. The method of claim 56, wherein said first dose comprises about 1×10⁶ cfu of mutant P. multocida.

58. The method of claim 31, wherein said second dose comprises from about 1×10² to about 1×10⁸ cfu of mutant P. multocida.

59. The method of claim 58, wherein said second dose comprises from about 1×10⁴ to about 1×10⁶ cfu of mutant P. multocida.

60. The method of claim 59, wherein said second dose comprises about 1×10⁶ cfu of mutant P. multocida.

61. The method of claim 46, wherein said first time point occurs before said poultry bird is hatched, and said first dose is administered in ovo.

62. The method of claim 61, wherein said first time point is between day 1 and day 26 of incubation of said poultry bird in ovo.

63. The method of claim 46, wherein said first time point is between 1 day of age and 10 weeks of age of said poultry animal.

64. The method of claim 63, wherein said first time point is between 4 weeks of age and 10 weeks of age of said poultry animal.

65. The method of claim 64, wherein said first time point is between 6 weeks of age and 8 weeks of age of said poultry animal.

66. The method of claim 46, wherein said second time point is between 1 day of age and 20 weeks of age of said poultry animal.

67. The method of claim 66, wherein said second time point is between 5 weeks of age and 20 weeks of age of said poultry animal.

68. The method of claim 67, wherein said second time point is between 12 weeks of age and 18 weeks of age of said poultry animal.
